(19) United States
(12) Patent Application Publication
(10) Pub. No.: US 2008/0070922 A1
(43) Pub. Date: Mar. 20, 2008

(54) Combination of Organic Compounds

(76) Inventors: Bryan Burkey, Winchester, MA (US); Thomas Hughes, Concord, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/576,860

(22) PCT Filed: Oct. 6, 2005
(86) PCT No.: PCT/US05/35917

§ 371 Date: Apr. 6, 2007

Related U.S. Application Data
(60) Provisional application No. 60/617,201, filed on Oct. 8, 2004.

(51) Int. Cl. A61K 31/496; A61K 31/40; A61P 15/00; A61P 17/00; A61P 27/02; A61P 3/04; A61P 3/08; A61P 3/10; A61P 5/00; A61P 9/00

(52) U.S. Cl. 514/252.18; 514/423

(57) ABSTRACT

The present invention relates to a combination, comprising; i) DPP IV inhibitor or a pharmaceutically acceptable salt thereof, and ii) at least one PDGF receptor tyrosine kinase inhibitor or a pharmaceutically acceptable salt thereof. The present invention furthermore relates to the use of such a combination for the prevention, delay of progression or treatment of a disease or condition selected from insulin resistance, impaired glucose metabolism (IGT), conditions of impaired glucose tolerance, conditions of impaired fasting plasma glucose, diabetes particularly type 1 or type 2 diabetes mellitus, obesity, diabetic retinopathy, macular degeneration, cataracts, diabetic nephropathy, glomerulosclerosis, diabetic neuropathy, erectile dysfunction, premenstrual syndrome, coronary heart disease, hypertension, angina pectoris, myocardial infarction, stroke, vascular restenosis, skin and connective tissue disorders, foot ulcerations and ulcerative colitis, endothelial dysfunction and impaired vascular compliance, and vascular events, cardiovascular morbidity or mortality associated with diabetes (e.g. type I or II) or IGT.

The invention relates to a combination, such as a combined preparation or pharmaceutical composition, respectively, comprising a DPP-IV inhibitor or a pharmaceutically acceptable salt thereof and at least one PDGF receptor tyrosine kinase inhibitor, or a pharmaceutically acceptable salt thereof.

It has now been found that a combination comprising at least one PDGF receptor tyrosine kinase inhibitor, e.g., as defined below, and a DPP-IV inhibitor as co-agent, e.g., as defined below, has a beneficial effect and is useful in the treatment of diseases or conditions which may be inhibited by PDGF receptor tyrosine kinase inhibition and conditions/disorders that might be treated by DPP-IV inhibition.

Thus in a first aspect, the present invention relates a combination, such as a combined preparation or pharmaceutical composition, respectively, comprising as active ingredients;
i) a DPP IV inhibitor or a pharmaceutically acceptable salt thereof, and
ii) a least one PDGF receptor tyrosine kinase inhibitor or a pharmaceutically acceptable salt thereof.

Preferably the combination is a pharmaceutical composition or a combined pharmaceutical preparation.

In this pharmaceutical composition, the combination partners (i) and (ii) can be administered together, one after the other or separately in one combined unit dosage form or in two separate unit dosage forms. The unit dosage form may also be a fixed combination.

The term “at least one therapeutic agent” shall mean that in addition to the DPP IV inhibitor one or more, for example two, furthermore three, active ingredients as specified according to the present invention can be combined.

The term “DPP-IV” as used herein is intended to mean dipeptidyl peptidase IV, also known as CD26. DPP-IV, a serine protease belonging to the group of post-proline/alanine cleaving amino-dipeptidases, specifically removes the two N-terminal amino acids from proteins having proline or alanine in position 2. DPP-IV can be used in the control of glucose metabolism because its substrates include the insulinotropic hormones glucagon like peptide-1 (GLP-1) and gastric inhibitory peptide (GIP). GLP-1 and GIP are active only in their intact forms; removal of their two N-terminal amino acids inactivates them.

In vivo administration of synthetic inhibitors of DPP-IV prevents N-terminal degradation of GLP-1 and GIP, resulting in higher plasma concentrations of these hormones, increased insulin secretion and, therefore, improved glucose tolerance.

The term “DPP-IV inhibitor” is intended to indicate a molecule that exhibits inhibition of the enzymatic activity of DPP-IV and functionally related enzymes, such as from 1-100% inhibition, and specially preserves the action of substrate molecules, including but not limited to GLP-1, GIP, peptide histidine methionine, substance P, neuropeptide Y, and other molecules typically containing alanine or proline residues in the second amino terminal position. Treatment with DPP-IV inhibitors prolongs the duration of action of peptide substrates and increases levels of their intact, undegraded forms leading to a spectrum of biological activities relevant to the disclosed invention.

For that purpose, chemical compounds are tested for their ability to inhibit the enzyme activity of purified CD26/DPP-IV. Briefly, the activity of CD26/DPP-IV is measured in vitro by its ability to cleave the synthetic substrate Gly-Pro-p-nitroanilide (Gly-Pro-pNA). Cleavage of Gly-Pro-pNA by DPP-IV liberates the product p-nitroanilide (pNA), whose rate of appearance is directly proportional to the enzyme activity. Inhibition of the enzyme activity by specific enzyme inhibitors slows down the generation of pNA. Stronger interaction between an inhibitor and the enzyme results in a slower rate of generation of pNA. Thus, the degree of inhibition of the rate of accumulation of pNA is a direct measure of the strength of enzyme inhibition. The accumulation of pNA is measured spectrophotometrically. The inhibition constant, Ki, for each compound is determined by incubating fixed amounts of enzyme with several different concentrations of inhibitor and substrate.

In the present context “a DPP-IV inhibitor” is also intended to comprise active metabolites and prodrugs thereof, such as active metabolites and prodrugs of DPP-IV inhibitors. An active “metabolite” is an active derivative of a DPP-IV inhibitor produced when the DPP-IV inhibitor is metabolized. A “prodrug” is a compound that is either metabolized to a DPP-IV inhibitor or is metabolized to the same metabolite(s) as a DPP-IV inhibitor.

DPP-IV inhibitors are known in the art. For example, DPP-IV inhibitors are in each case generically and specifically disclosed e.g. in WO 98/19998,DE19616 486 A1, WO 00/34241, WO 95/15309, WO 01/72290, WOO1/52825, WO 9310127, WO 9925719, WO 9938501, WO 9946272, WO 9967278 and WO 9967279.

Preferred DPP-IV inhibitors are described in the following patent applications; WO 02053548 especially compounds 1001 to 1293 and examples 1 to 124, WO 02067918 especially compounds 1000 to 1278 and 2001 to 2159, WO 02066627 especially the described examples, WO 02/068420 especially all the compounds specifically listed in the examples I to LXIII and the described corresponding analogues, even preferred compounds are 2(28), 2(88), 2(119), 2(136) described in the table reporting IC50, WO 02083128 especially examples 1 to 13, US 2003096846 especially the specifically described compounds, WO 2004/037181 especially examples 1 to 33, WO 0168603 especially compounds of examples 1 to 109, EP1258480 especially compounds of examples 1 to 60, WO 0181337 especially examples 1 to 118, WO 02083109 especially examples 1A to 1D, WO 030003250 especially compounds of examples 1 to 166, most preferably 1 to 8, WO 03035067 especially the compounds described in the examples, WO 03/035057 especially the compounds described in the examples, US2003216450 especially examples 1 to 450, WO 99/46272 especially compounds of claims 12, 14, 15 and 17, WO 0197808 especially compounds of claim 2, WO 03002553 especially compounds of examples 1 to 33, WO 01/34594 especially the compounds described in the examples 1 to 4, WO 02051836 especially examples 1 to 712, EP1245568 especially examples 1 to 7, EP1258476 especially examples 1 to 32, US 2003087950 especially the described examples, WO 02/076450 especially examples 1 to 128, WO 03000180 especially examples 1 to 162, WO 03000181 especially examples 1 to 66, WO 03004498 especially examples 1 to 33, WO 0302942 especially examples 1 to 68, US 6482844 especially the described examples, WO 0155105 especially the compounds listed in the examples 1 and 2, WO 0202560 especially examples 1 to 166, WO 03004496 especially examples 1 to 103, WO 03/024965 especially examples 1 to 54, WO 0303727 especially examples 1 to 209, WO 0368757 especially examples 1 to 88, WO 03074500 especially examples 1 to 72, examples 4.1 to 4.23, examples 5.1 to 5.10, examples 6.1 to 6.30, examples 7.1 to 7.23, examples 8.1 to 8.10, examples 9.1 to 9.30, WO 02038541 especially examples 1 to 53, WO 02062764 especially examples 1 to 293, preferably the compound of example 95 (2-{{3-(Aminomethyl)-4-butoxy-2-neopentyl-1-oxo-1,2 dihydro-6-isoquinolinyl}oxy}acetamide hydrochloride), WO 02308090 especially examples 1-1 to 1-109, examples 2-1 to 2-9, example 3, examples 4-1 to 4-19, examples 5-1 to 5-39, examples 6-1 to 6-4, examples 7-1 to 7-10, examples 8-1 to 8-8, examples 7-1 to 7-7 of page 90, examples 8-1 to 8-59 of pages 91 to 95, examples 9-1 to 9-33, examples 10-1 to 10-20, US 2003225102 especially compounds 1 to 115, compounds of examples 1 to 121,preferably compounds a) to z), aa) to az), ba) to bz), ca) to cz) and da) to dk), WO 0214271 especially examples 1 to 320 and US 2003096857 and WO 2004/052850 especially the specifically described compounds such as examples 1 to 42 and compounds of claim 1, DE 102 56 264 A1 especially the described compounds such as examples 1 to 181 and the compounds of claim 5, WO 04/076433 especially the compounds specifically described, such as listed in table A, preferably the compounds listed in table B, preferably compounds I to XXXXVII, or compounds of claims 6 to 49, WO 04/071454 especially the specifically described compounds e.g. compounds 1 to 53 or compounds of tables Ia to If, or compounds of claims 2 to 55, WO 02/068420,especially the compounds specifically described, such as the compounds I to LXIII or Beispiele I and analogues 1 to 140 or Beispiele 2 and analogues 1 to 174 or Beispiele 3 and analogues 1, or Beispiele 4 to 5, or Beispiele 6 and analogues 1 to 5, or Beispiele 7 and analogues 1-3, or Beispiele 8 and analogue 1, or Beispiele 9, or Beispiele 10 and analogues 1 to 531 even preferred are compounds of claim 13, WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965024942 especially the compounds specifically described, such compounds 1 to 54, Wo03002593 especially the compounds specifically described, such compounds table 1 or of claims 2 to 15, WO03037327 especially the compounds specifically described, such compounds of examples 1 to 209 WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965024942 especially the compounds specifically described, such compounds 1 to 54, Wo03002593 especially the compounds specifically described, such compounds table 1 or of claims 2 to 15, WO03037327 especially the compounds specifically described, such compounds of examples 1 to 209, WO0238541, WO0230890.

WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965 especially the compounds specifically described, such compounds 1 to 54, WO 03002593 especially the compounds specifically described, such compounds table 1 or of claims 2 to 15, WO03037327 especially the compounds specifically described, such compounds of examples 1 to 209, WO0238541 especially the compounds specifically described, such compounds of examples 1 to 53, WO 03/002531 especially the compounds specifically described preferably the compounds listed on page 9 to 13, most preferably the compounds of examples 1 to 46 and even preferred compound of example 9, U.S. Pat. No. 6,395,767 preferably compound of examples 1 to 109 most preferably compound of example 60, U.S. application Ser. No. 09/788,173 filed Feb. 16, 2001 (attorney file LA50) especially the described examples, WO99/38501 especially the described examples, WO99/46272 especially the described examples and DE19616 486 A1 especally val-pyr, val-thiazolidide, isoleucyl-thiazolidide, isoleucyl-pyrrolidide, and fumar salts of isoleucyl-thiazolidide and isoleucyl-pyrrolidide.

Further preferred DPP-IV inhibitors include the specific examples disclosed in U.S. Pat. No. 6,124,305 and U.S. Pat. No. 6,107,317, International Patent Applications, Publication Numbers WO 95153 09 and WO 9818763.

In each case in particular in the compound claims and the final products of the working examples, the subject matter of the final products, the pharmaceutical preparations and the claims are hereby incorporated into the present application by reference to these publications.

Published patent application WO 9819998 discloses N—(N′-substituted glycyl)-2-cyano pyrrolidines, in particular 1-[2-[5-Cyanopyridin-2-yl]amino]- ethylamino]acetyl-2-cyano-(S)-pyrrolidine (NVP-DPP728).

Published patent application WO 0034241 and published patent U.S. Pat. No. 6,110,949 disclose N-substituted adamantyl-amino-acetyl-2-cyano pyrrolidines and W (substituted glycyl)-4-cyano pyrrolidines respectively. DPP-IV inhibitors of interest are specially those cited in claims 1 to 4. In particular these applications describe the compound 1-[[(3-Hydroxy-1-adamantyl) amino]acetyl]-2-cyano-(S)-pyrrolidine (also known as LAF237 or vildagliptin).

Published patent application WO 9515309 discloses amino acid 2- cyanopyrrolidine amides as inhibitors of DPP-IV Published patent application WO 9529691 discloses peptidyl derivates of diesters of alpha-aminoalkylphosphonic acids, particularly those with proline or related structures. DPP-IV inhibitors of interest are specially those cited in Table 1 to 8.

In WO 01/72290 DPP-IV inhibitors of interest are specially those cited in example 1 and claims 1, 4, and 6.

WO01/52825 specially discloses (S)-1-{2-[5-cyanopyridin-2yl)amino]ethyl-aminoacetyl) -2-cyano-pyrrolidine or (S)-1-[(3-hydroxy-1-adamantyl)amino]acetyl-2-cyano-pyrrolidine.

Published patent application WO 9310127 discloses proline boronic esters useful as DPP-IV inhibitors. DPP-IV inhibitors of interest are specially those cited in examples 1 to 19.

Published patent application WO 9925719 discloses sulphostin, a DPP-IV inhibitor prepared by culturing a Streptomyces microorganism.

Published patent application WO 9938501 discloses N-substituted 4-8 membered heterocyclic rings. DPP-IV inhibitors of interest are specially those cited in claims 15 to 20.

Published patent application WO 9946272 discloses phosphoric compounds as inhibitors of DPP-IV. DPP-IV inhibitors of interest are specially those cited in claims 1 to 23.

Published patent applications WO 9967278 and WO 9967279 disclose DPP-IV prodrugs and inhibitors of the form A-B-C where C is either a stable or unstable inhibitor of DPP-IV.

Other preferred DPP-IV inhibitors are the compounds of formula I, II or III disclosed in the patent application WO 03/057200 on page 14 to 27. Most preferred DPP-IV inhibitors are the compounds specifically described on pages 28 and 29.

Any of the substances disclosed in the above mentioned patent documents, hereby included by reference, are considered potentially useful as DPP-IV inhibitors to be used in carrying out the present invention.

In a further preferred embodiment, the DPP-IV inhibitor is a N-peptidyl-O-aroyl hydroxylamine or a pharmaceutically acceptable salt thereof. Aroyl is, for example, naphthylcarbonyl; or benzoyl which is unsubstituted or mono- or disubstituted, for example, by lower alkoxy, lower alkyl, halogen or, preferably, nitro. The peptidyl moiety comprises preferably two α-amino acids, e.g. glycine, alanine, leucine, phenylalanine, lysine or proline, of which the one attached directly to the hydroxylamine nitrogen atom is preferably proline.

Preferably, the N-peptidyl-O-aroyl hydroxylamine is a compound of formula VII 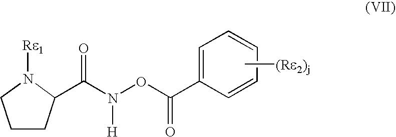
wherein

j is 0, 1 or 2;

R_{ε}_{₁} represents the side chain of a natural amino acid; and

R_{ε}_{₂} represents lower alkoxy, lower alkyl, halogen or nitro;

or a pharmaceutically acceptable salt thereof.

In a very preferred embodiment of the invention, the N-peptidyl-O-aroyl hydroxylamine is a compound of formula VIIa 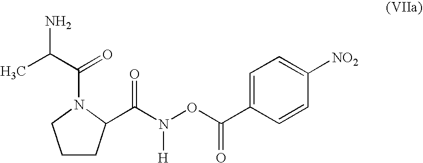
or a pharmaceutically acceptable salt thereof.

N-Peptidyl-O-aroyl hydroxylamines, e.g. of formula VII or VIIa, and their preparation are described by H. U. Demuth et al. in J. Enzyme Inhibition 1988, Vol. 2, pages 129-142, especially on pages 130-132.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl) amino]ethylamino]acetyl-2-cyano-(S)-pyrrolidine and pharmaceutical salts thereof.

Preferred DPP-IV inhibitors are those described by Mona Patel and col. (Expert Opinion Investig Drugs. 2003 April; 12(4):623-33) on the paragraph 5, especially P32/98, K-364, FE-999011, BDPX, NVP-DDP-728 and others, which publication is hereby incorporated by reference especially the described DPP-IV inhibitors.

Another preferred inhibitor is the compound BMS-477118 disclosed in WO 2001068603 or U.S. Pat. No. 6,395,767 (compound of example 60) also known as is (1S,3S,5S)-2-[(2S)-2-amino-2-(3-hydroxytricyclo[3.3.1.1^{3.7}]dec-1-yl)-1-oxoethyl]-2-azabicyclo[3.1.0]hexane-3-carbonitrile, benzoate (1:1) as depicted in Formula M of the patent application WO 2004/052850 on page 2, and the corresponding free base, (IS,3S,5S)-2-[(2S)-2-amino-2-(3-hydroxy-tricyclo[3.3.1.1^{3.7}]dec-1-yl)-1-oxoethyl]-2-azabicyclo-[3.1.0]hexane-3-carbonitrile (M′) and its monohydrate (M″) as depicted in Formula M of the patent application WO 2004/052850 on page 3. The compound BMS-477118 is also known as saxagliptin.

Another preferred inhibitor is the compound GSK23A disclosed in WO 03/002531 (example 9) also known as (2S,4S)-1-((2R)-2-Amino-3-[(4-methoxybenzyl)sulfonyl]-3-methylbutanoyl)-4-fluoropyrrolidine-2-carbonitrile hydrochloride.

FE-999011 is described in the patent application WO 95/15309 page 14, as compound No. 18.

P32/98 or P3298 (CAS number: 251572-86-8) also known as 3-[(2S,3S)-2-amino-3-methyl-1-oxopentyl]thiazolidine can be used as 3-[(2S,3S)-2-amino-3-methyl-1-oxopentyl]thiazolidine and (2E)-2-butenedioate (2:1) mixture such as shown below 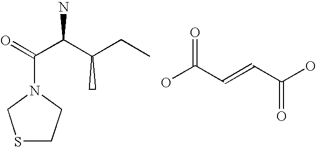
and is described in WO 99/61431 and also in Diabetes 1998, 47, 1253-1258, in the name of Probiodrug, as well as the compound P93/01 described by the same company.

Other very preferred DPP-IV inhibitors of the invention are described in the International patent application WO 02/076450 (especially the examples 1 to 128) and by Wallace T. Ashton (Bioorganic & Medicinal Chemistry Letters 14 (2004) 859-863 ) especially the compound 1 and the compounds listed in the tables 1 and 2. The preferred compound is the compound 21e (table 1) of formula:

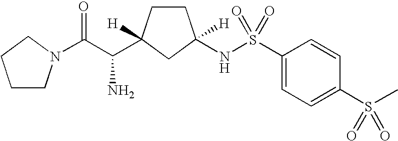

Other preferred DPP-IV inhibitors are described in the patent applications WO 2004/037169 especially those described in the examples 1 to 48 and WO 02/062764 especially the described examples 1 to 293, even preferred are the compounds 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide described on page 7 and also in the patent application WO2004/024184 especially in the reference examples 1 to 4.

Other preferred DPP-IV inhibitors are described in the patent application WO 03/004498 especially examples 1 to 33 and most preferably the compound of the formula 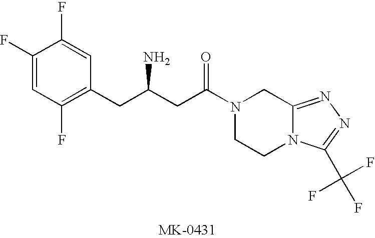
described by the example 7 and also known as MK-0431 or Sitagliptin.

Preferred DPP-IV inhibitors are also described in the patent application WO 2004/037181 especially examples 1 to 33 and most preferably the compounds described in the claims 3 to 5.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl) amino]ethylamino]acetyl-2-cyano-(S)-pyrrolidine, MK-431 and pharmaceutical salts thereof.

Most preferred DPP-IV inhibitors are selected from [S]-1-[2-(5-cyano-2-pyridinylamino)ethylamino]acetyl-2-pyrrolidine carbonitrile monohydrochloride, (S)-1-[(3-hydroxy-1-adamantyl)amino]acetyl-2-cyano-pyrrolidine and L-threo-isoleucyl thiazolidine (compound code according to Probiodrug: P32/98 as described above), MK-0431, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy)acetamide and optionally pharmaceutical salts thereof.

Especially preferred are 1-{2-[(5-cyanopyridin-2-yl) amino]ethylamino}acetyl-2(S)-cyano-pyrrolidine dihydrochloride (DPP728) (also named [S]-1-[2-(5-cyano-2-pyridinylamino)ethylamino]acetyl-2-pyrolidine carbonitrile monohydrochloride), of formula 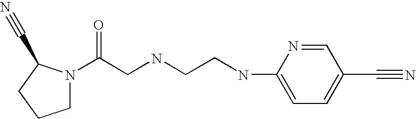
especially the dihydrochloride and monohydrochloride thereof, and 1-[(3-hydroxy-1-adamantyl) amino]acetyl-2-cyano-, (S) (also named (S)-1-[(3-hydroxy-1-adamantyl)amino]acetyl-2-cyano-pyrrolidine, LAF237 or vildagliptin) of formula 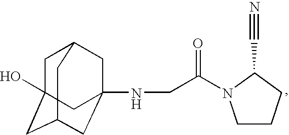
and L-threo-isoleucyl thiazolidine (compound code according to Probiodrug: P32/98 as described above), MK-0431, GSK23A, saxagliptin, 3-(aminomethyl)-2-isobuthyl-1-oxo-4- phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally pharmaceutical salts thereof.

DPP728 and vildagliptin are specifically disclosed in Example 3 of WO 98/19998 and Example 1 of WO 00/34241, respectively. The DPP-IV inhibitor P32/98 (see above) is specifically described in Diabetes 1998, 47, 1253-1258. DPP728 and LAF237 can be formulated as described on page 20 of WO 98/19998 or in WO 00/34241 or in the International Patent Application No. EP2005/000400 (application number).

Especially preferred are orally active DPP-IV inhibitors.

Any of the substances disclosed in the above mentioned patent documents or scientific publications, hereby included by reference, are considered potentially useful as DPP-IV inhibitors to be used in carrying out the present invention.

In each case in particular in the compound claims and the final products of the working examples, the subject matter of the final products, the pharmaceutical preparations and the claims are hereby incorporated into the present application by reference to these publications.

DPP-IV inhibitor to be used alone according to the present invention can be used in association with a carrier.

Especially preferred are orally active DPP-IV inhibitors.

In each case in particular in the compound claims and the final products of the working examples, the subject matter of the final products, the pharmaceutical preparations and the claims are hereby incorporated into the present application by reference to these publications.

The term “at least one” shall mean that in addition to the renin inhibitor one or more, for example two, furthermore three, active ingredients as specified according to the present invention can be combined.

The PDGF-R- , tyrosine kinase inhibitors used according to the present invention are preferably selected from the group comprising the following compounds: 4-(4-methylpiperazin-1-ylmethyl)-N-[4-methyl-3-(4-pyridin-3-yl)pyrimidin-2-ylamino)phenyl]-benzamide, 4-Methyl-N-[3-(4-methyl-imidazol-1-yl)-5-trifluoromethyl-phenyl]-3-(4-pyridin-3-pyrimidin-2-ylamino)-benzamide, an inhibitor of PDGF-receptor isoforms, compounds as described in Mahboobi S et al., J. Med. Chem. 2002, 45:1002-1018 and hereby incorporated by reference; the PDGF receptor kinase blocker AG1295 having the CAS Number 71897-07-9; AG1295/96 as described by Kovalenko M et al., Cancer Res. 1994 54:6106-6114 and Ludewig D et al., Cell Tissue Res. 2000, 299:97-103 and hereby incorporated by reference; CT52923 (4-(6,7-dimethoxy-4-quinazolinyl)-N-(3,4-methylenedioxybenzyl)-1-piperazinethiocarboxamide); RP-1776; GFB-111; pyrrolo[3,4-c]-beta-carboline-diones, SU 102 (developed by SUGEN); AG1296 having the CAS Number 146535-11-7; RPR101511A developed by Aventis Pharma; CDP 860 and Zvegf3 developed by ZymoGenetics; CP 673451 and PD 170262 from Pfizer; KI 6783, having the CAS number 190726-45-5, an inhibitor of PDGF-R developed by Kirin Brewery, Japan; KN 1022 developed by Kyowa Hakko in Japan and Millenium Pharmaceuticals in US; AG 13736 developed by Pfizer; CHIR 258 developed by Chiron Corporation; MLN 518 from Millenium Pharmaceuticals and SU 11248 from SUGEN-Pfizer, Leflunomide; or pharmaceutically acceptable salts thereof.

CT52923 has been described by Matsuno K, et al., “Synthesis and structure activity relationships of PDGF receptor phosphorylation inhibitor-1.” in 18th Symposium on Medicinal Chemistry; 1998 Nov. 25-27;Kyoto, Japan, the Pharmaceutical Society of Japan, Division of Medicinal Chemistry, Tokyo, Japan: Abstract 2-P-05.

RP-1776, a cyclic peptide, was isolated from the culture broth of Streptomyces sp. KY11784. It is described, e.g. by Toki S, Agatsuma T, et al., J. Antibiot. (Tokyo) 2001 May; 54(5):405-14.

GFB-111 is described, e.g. in Blaskovich M A et al., Nat. Biotechnol. 2000 October; 18(10):1065-70 and in Delarue F. et al, 91^{st} Annual meeting of the American Association for Cancer research, 41:458, 2000.

Pyrrolo[3,4-c]-beta-carboline-diones is described, e.g. by Teller S, Eur. J. Med. Chem. 2000 April; 35(4):413-27.

CDP 860 is a pegylated antibody fragment derived from the human anti-platelet derived growth factor beta receptor antibody.

PD 170262 or 2-[4-(2-diethlaminoethoxy)phenylamino]-8-methyl-6-(3-thienyl) pyrido[2,3-d]pyrimidin-7(8H)-one is a potent inhibitor of tyrosine kinase with selectivity for the platelet—derived growth factor tyrosine kinase. Synthesis and tyrosine kinase inhibitory activity of a series of 2-amino-8H-pyrido[2,3-d]pyrimidines is described, e.g. in Klutchko S. et al., 213^{th} American Chemical Society National meeting: abst. MEDI 201 (poster), 1997, USA.

KI 6783 or 4-(3,4-dimethoxyphenoxy)-6,7-dimethoxyquinoline is described, e.g. in Kubo K. et al, Bioorganic and Medicinal Chemistry Letters 7:2935-2940, 1997 and Yagi M. et al., Exp. Cell Research 234:285-92, 1997.

KN1022 or 6,7-dimethoxy-4-[4-(4-nitrophenyl)aminocarbonylpiperazin-1yl]-quinazoline, which inhibits PDGFR phosphorylation, is described, e.g. in 217^{th} American Chemical Society National meeting abstr. MEDI 061, Part1, 1999, Japan.

AG 013736 or N-methyl-2-[3-[2-(2-pyridyl)vinyl]-1H-indazole-6-ylsulfanyl]-benzamide is disclosed, e.g. in Heller et al., Pharmacological activities of AG 013736, a small molecule inhibitor of VEGF/PDGFR tyrosine kinases, 93^{rd} Annual meeting f the American association for Cancer research 43:1082, 2002, USA.

CHIR 258 is an orally active amino-benzimidazole quinoline growth factor kinase inhibitor which demonstrated a spectrum of inhibitory activity against receptor tyrosine kinases, e.g. from the PDGFR family. CHIR 258 is disclosed, e.g. in Steigerwalt R et al. and Lee S H et al. in 94^{th} Annual Meeting of the American Association for Cancer Research 753 (plus poster) abstr. 3783 and 934 (plus poster) abstr. R4702, respectively, 2003, USA.

SU 11248 or 5-[3-fluoro-2-oxo-1,2-dihydroindol-(3Z)-ylidenemethyl]-2,4-dimethyl-1H-pyrrole-3-carboxylic acid(2-diethylaminoethyl)amine is multiple targeted kinase inhibitor with selectivity for, e.g. PDGFR. SU11248 is disclosed, e.g. in Xin L. et al., 93^{rd} Annual Meeting of the American Association for Cancer Research 43:1081 (plus poster), 2002, USA.

MLN 518 is a piperazinyl derivative of quinazoline of formula 4-[4-(N-para-iso-propoxyphenylcarbamoyl)-1-piperazinyl]-6-methoxy-7-(piperidinopropyloxy)-quinazoline that inhibits, e.g. PDGF R phosphorylation in binding assays, it is described, e.g. by Stone R M et al., Blood 102:65-66, 2003, Kelly L M et al., Cancer Cell 1: 421-23, 2002.

Leflunomide (SU 101) or 4-Isoxazolecarboxamide, 5-methyl-N-[4-(trifluoromethyl)phenyl] is a tyrosine kinase inhibitor.

Preferred PDGF receptor tyrosine kinase inhibitors are N-phenyl-2-pyrimidine-amine derivatives of formula II 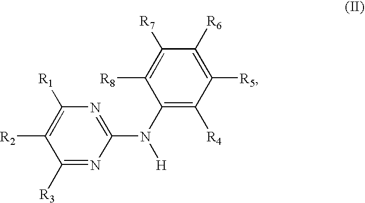
as described in the patent applications EP 0 564 409 A1 and WO 99/03854, incorporated into the present application by reference.

Preference is given above all especially to the compound of formula (II) which is CGP 57148B {N-{5-[4-(4-methyl-piperazino-methyl)-benzoylamido]-2-methylphenyl)-4-(3-pyridyl)-2-pyrimidine-amine}. CGP 57148B (hereinafter: “Imatinib” [International Non-proprietary Name]) and the use thereof, especially as an anti-tumour agent, are described in Example 21 of European patent application EP-A-0 564 409, which was published on 6 Oct. 1993, and in equivalent applications and patents in numerous other countries, e.g. in U.S. Pat. No. 5,521,184 and in Japanese patent 2706682. Another preference is given to the β-crystal form of 4-(4-methylpiperazin-1-ylmethyl)-N-[4-methyl-3-(4-pyridin-3-yl) pyrimidin-2-ylamino)phenyl]-benzamide methanesulfonate as described in the European patent application No. 998 473 published on May 10, 2000.

The term “4-(4-methylpiperazin-1-ylmethyl)-N-[4-methyl-3-(4-pyridin-3-yl)pyrimidin-2-yl-amino)phenyl]-benzamide” includes all crystal forms especially the β-crystal form as described in the European patent application No. 998 473.

Very preferably a N-phenyl-2-pyrimidine-amine derivative of formula (II) is used in the form of its monomesylate salt.

The compounds of formula II are generically and specifically disclosed in the patent applications EP 0 564 409 A1 and WO 99/03854, in particular in the compound claims and the final products of the working examples, the subject-matter of the final products, the pharmaceutical preparations and the claims are hereby incorporated into the present application by reference to these publications. Comprised are likewise the corresponding stereoisomers as well as the corresponding polymorphs, e.g. crystal modifications, which are disclosed therein.

In EP 0 564 409 A1 the compounds II are described to be useful for the therapy of cancer, thrombosis, psoriasis, fibrosis, dermatosclerosis and atherosclerosis.

For the purposes of isolation or purification, as well as in the case of compounds that are used further as intermediates, it is also possible to use pharmaceutically unacceptable salts. Only pharmaceutically acceptable, non-toxic salts are used for therapeutic purposes, however, and those salts are therefore preferred.

Further suitable PDGF receptor tyrosine kinase inhibitors are disclosed in WO 98/35958, especially the compound of Example 62, and U.S. Pat. No. 5,093,330 in each case in particular in the compound claims and the final products of the working examples, the subject-matter of which are hereby incorporated into the present application by reference to these publications.

Other preferred compounds are described in the patent application WO 04/005281, especially the examples, most preferably the compound of example 92 of formula 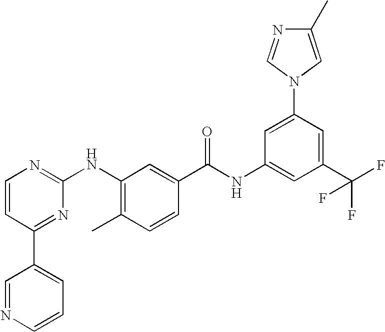
which is also known as 4-Methyl-N-[3-(4-methyl-imidazol-1-yl)-5-trifluoromethyl-phenyl]-3-(4-pyridin-3-yl-pyrimidin-2-ylamino)-benzamide.

Preferred PDGF receptor tyrosine kinase inhibitors are selected from 4-(4-methylpiperazin-1-ylmethyl)-N-[4-methyl-3-(4-pyridin-3-yl)pyrimidin-2-ylamino)phenyl]-benzamide (imatinib), 4-(4-methylpiperazin-1-ylmethyl)-N-[4-methyl-3-(4-pyridin-3-yl)pyrimidin-2-ylamino)phenyl]-benzamide methanesulfonate, 4-Methyl-N-[3-(4-methyl-imidazol-1-yl)-5-trifluoromethyl-phenyl]-3-(4-pyridin-3-yl-pyrimidin-2-ylamino)-benzamide, CT52923 (4-(6,7-dimethoxy-4-quinazolinyl)-N-(3,4-methylenedioxybenzyl)-1-piperazinethiocarboxamide), RP-1776, GFB-111, pyrrolo[3,4-c]-beta-carboline-diones, SU 102 (developed by SUGEN), AG1296 (CAS Number 146535-11-7), AG1296 (CAS Number 71897-07-9) and RPR101511A or, in each case, a pharmaceutically acceptable salt thereof.

In each case where appropriate, e.g. if the compound is not present as a pharmaceutically acceptable salt per se as in the case of hydrochlorothiazide, these compounds also include their pharmaceutically acceptable salts.

The corresponding active ingredients or a pharmaceutically acceptable salts thereof may also be used in form of a solvate, such as a hydrate or including other solvents, used for crystallization.

The most preferred PDGF receptor tyrosine kinase inhibitors are N-{5-[4-(4-methyl-piperazino-methyl)-benzoylamido]-2-methylphenyl}-4-(3-pyridyl)-2-pyrimidine-amine (imatinib) and 4-Methyl-N-[3-(4-methyl-imidazol-1-yl)-5-trifluoromethyl-phenyl]-3-(4-pyridin-3-yl-pyrimidin-2-ylamino)-benzamide or in each case a pharmaceutically acceptable salt thereof such as the mono-hydrochloride.

Preferred are combinations, such as combined preparations or pharmaceutical compositions, respectively, comprising a DPP-IV inhibitor preferably LAF237 or a pharmaceutically accepted salt thereof and as second active agent an active agent selected from the group consisting of 4-(4-methylpiperazin-1-ylmethyl)-N-[4-methyl-3-(4-pyridin-3-yl)pyrimidin-2-ylamino)phenyl]-benzamide (imatinib), 4-(4-methylpiperazin-1-ylmethyl)-N-[4-methyl-3-(4-pyridin-3-yl)pyrimidin-2-ylamino)phenyl]-benzamide methanesulfonate, CT52923 (4-(6,7-dimethoxy-4-quinazolinyl)-N-(3,4-methylenedioxybenzyl)-1-piperazinethiocarboxamide), 4-Methyl-N-[3-(4-methyl-imidazol-1-yl)-5-trifluoromethyl-phenyl]-3-(4-pyridin-3-yl-pyrimidin-2-ylamino)-benzamide, RP-1776, GFB-111, pyrrolo[3,4-c]-beta-carboline-diones, SU 102 (developed by SUGEN), AG1296 (CAS Number 146535-11-7), AG1296 (CAS Number 71897-07-9) and RPR101511A, or in each case, a pharmaceutically acceptable salt thereof.

The corresponding active ingredients or a pharmaceutically acceptable salt thereof may also be used in form of a solvate, such as a hydrate or including other solvents, used for crystallization.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases.

All of these marketed products may be utilized in as such for combination therapy according to the present invention.

The structure of the active agents identified by generic or tradenames may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

All the more surprising is the experimental finding that the combined administration of a DPP IV inhibitor or a salt thereof and at least one PDGF receptor tyrosine kinase inhibitor results not only in a beneficial, especially a synergistic, therapeutic effect, but also in additional benefits resulting from the combined treatment and further surprising beneficial effects compared to a monotherapy applying only one of the pharmaceutically active compounds used in the combinations disclosed herein.

It can be shown by established test models and especially those test models described herein that the combination of the DPP-IV inhibitor with at least one PDGF receptor tyrosine kinase inhibitor results in a more effective prevention or preferably treatment of diseases specified in the following. In particular, it can be shown by established test models and especially those test models described herein that the combination of the present invention results in a more effective prevention or preferably treatment of diseases specified hereinafter.

If taken simultaneously, this results not only in a further enhanced beneficial, especially a synergistic, therapeutic effect, but also in additional benefits resulting from the simultaneous treatment such as a surprising prolongation of efficacy, a broader variety of therapeutic treatment and surprising beneficial effects on pancreatic Beta-cells, diabetes such as type I or type II diabetes, IGT, obesity and vascular events, cardiovascular morbidity or mortality associated with diabetes or IGT, for a number of combinations as described herein.

The term “potentiation” shall mean an increase of a corresponding pharmacological activity or therapeutical effect, respectively. Potentiation of one component of the combination according to the present invention by co-administration of another component according to the present invention means that an effect is being achieved that is greater than that achieved with one component alone.

The term “synergistic” shall mean that the drugs, when taken together, produce a total joint effect that is greater than the sum of the effects of each drug when taken alone.

Moreover, for a human patient, especially for elderly people, it is more convenient and easier to remember to take two tablets at the same time, e.g. before a meal, than staggered in time, i.e. according to a more complicated treatment schedule. More preferably, both active ingredients are administered as a fixed combination, i.e. as a single tablet, in all cases described herein. Taking a single tablet is even easier to handle than taking two tablets at the same time. Furthermore, the packaging can be accomplished with less effort.

The person skilled in the pertinent art is fully enabled to select a relevant and standard animal test model to prove the hereinbefore and hereinafter indicated therapeutic indications and beneficial effects.

The pharmaceutical activities as effected by administration of the combination of the active agents used according to the present invention can be demonstrated e.g. by using corresponding pharmacological models known in the pertinent art.

The insulin secretion enhancing properties of the combination according to the present invention may be determined by following the methodology as disclosed, for example, in the publication of T. Ikenoue et al. Biol. Pharm. Bull. 29(4), 354-359 (1997).

The corresponding subject matter of these references is herewith incorporated by reference in this specification.

Accordingly, the combination according to the present invention may be used, e.g., for the prevention, delay of progression or treatment of diseases and disorders that may be inhibited by DPP IV inhibition and/or by inhibiting the PDGF tyrosine kinase receptor.

Thus in a further aspect the present invention concerns the use of a combination comprising
i) a DPP IV inhibitor or a pharmaceutically acceptable salt thereof, and
ii) at least one PDGF receptor tyrosine kinase inhibitor, or a pharmaceutically acceptable salt thereof
   for the manufacture of a medicament for the prevention, delay of progression or treatment of diseases and disorders that may be inhibited by DPP IV inhibition and/or by inhibiting the PDGF tyrosine kinase receptor.

The invention furthermore relates to a method for the prevention of, delay of progression of, treatment of diseases and disorders that may be inhibited by DPP IV inhibition and/or by inhibiting the PDGF tyrosine kinase receptor, comprising administering to a warm-blooded animal, including man, in need thereof a jointly effective amount of a combination of a DPP IV inhibitor or a pharmaceutically acceptable salt thereof with at least one therapeutic agent selected from an agent interacting with a PDGF receptor tyrosine kinase inhibitor, or a pharmaceutically acceptable salt thereof; and at least one additional pharmaceutically acceptable carrier.

The invention furthermore relates to a pharmaceutical composition for the prevention of, delay of progression of, treatment of a disease or condition selected from diseases and disorders that may be inhibited by DPP IV inhibition and/or by inhibiting the PDGF tyrosine kinase receptor, comprising a combination of a DPP IV inhibitor or a pharmaceutically acceptable salt thereof with at least one PDGF receptor tyrosine kinase inhibitor, or a pharmaceutically acceptable salt thereof; and at least one additional pharmaceutically acceptable carrier.

Methods or uses as described above, wherein the disease or condition is selected from insulin resistance, impaired glucose metabolism (IGT), conditions of impaired glucose tolerance, conditions of impaired fasting plasma glucose, diabetes particularly type 1 or type2 diabetes mellitus, obesity, diabetic retinopathy, macular degeneration, cataracts, diabetic nephropathy, glomerulosclerosis, diabetic neuropathy, erectile dysfunction, premenstrual syndrome, coronary heart disease, hypertension, angina pectoris, myocardial infarction, stroke, vascular restenosis, skin and connective tissue disorders, foot ulcerations and ulcerative colitis, endothelial dysfunction and impaired vascular compliance, and vascular events, cardiovascular morbidity or mortality associated with diabetes (e.g. type I or II) or IGT.

Most preferably, the disease or condition is selected from obesity, diabetes (type 1 or type 2 diabetes), IGT and vascular events, cardiovascular morbidity or mortality associated with diabetes (e.g. type I or II) or IGT.

Preferred combinations for the described uses or methods are described herein.

A “disease or condition which may be inhibited by a DPP-IV inhibitor” as defined in this application comprises, but is not limited to insulin resistance, impaired glucose metabolism, conditions of impaired glucose tolerance, conditions of impaired fasting plasma glucose, diabetes particularly type 2 diabetes mellitus, obesity, diabetic retinopathy, macular degeneration, cataracts, diabetic nephropathy, glomerulosclerosis, diabetic neuropathy, erectile dysfunction, premenstrual syndrome, coronary heart disease, hypertension, angina pectoris, myocardial infarction, stroke, vascular restenosis, skin and connective tissue disorders, foot ulcerations and ulcerative colitis, endothelial dysfunction, impaired vascular compliance. This definition also includes beneficial effects on diseases and conditions associated with diabetes or IGT (e.g. less gain of weight or less vascular events and cardiovascular morbidity or mortality).

“vascular events” associated with diabetes as defined in this application comprises, but is not limited to atherosclerosis; thrombosis; cerebrovascular diseases such as stroke, ischemia, stroke related mortality and stroke related dementia; peripheral arterial disease such as limb ischemia, claudation or intermittent claudation, undergo amputation; microvascular diseases and sequelae such as neuropathy, nephropathy and retinopathy, macrovascular diseases such as myocardial infarction, other coronary artery diseases; cardiac hypertrophy.

Preferably, a “disease or condition which may be inhibited by a DPP-IV inhibitor” is selected from impaired glucose metabolism, conditions of impaired glucose tolerance (IGT), conditions of impaired fasting plasma glucose, diabetes particularly type 1 or type 2 diabetes, obesity, diabetic retinopathy, diabetic nephropathy, diabetic neuropathy, foot ulcerations and vascular events, cardiovascular morbidity or mortality associated with diabetes or IGT.

A “disease or condition which may be inhibited by PDGF receptor tyrosine kinase inhibitor” is preferably selected from malignant or non-malignant proliferative disorders such as Chronic Myeloid Leukemia, Philadelphia Chromosome positive acute leukemia and Acute Myeloid Leukemia inhibition of angiogenesis; tumors [such as leukemias, gliomas, sarcomas; tumours of prostate, colon, breast, lung, or ovary], atherosclerosis, thrombosis [in general: disorders of smooth muscle cells of blood vessels]; sclerodermitis; psoriasis, restenosis, fibrosis; hepatic fibrosis or pneumonitis; asthma, prevention of transplantation induced disorders such as obliterative bronchiolitis; prevention of cell invasion by certain bacteria, like Porphyromonas gingivalis; multi-drug resistance or Hypereosinophilic syndrome; gastrointestinal stromal tumours (GIST); autoimmune diseases more particularly selected from the group consisting of multiple sclerosis, ulcerative colitis, Crohn's disease, rheumatoid arthritis and polyarthritis, scleroderma, lupus erythematosus, dermatomyositis, pemphigus, polymyositis, vasculitis, as well as graft-versus host diseases; inflammatory diseases more particularly inflammatory diseases associated with mast cells, such as rheumatoid arthritis, conjunctivitis, rheumatoid spondylitis, osteoarthritis, gouty arthritis and other arthritic conditions; diabetic nephropathy, but also of glomerulonephritis, chronic pyelonephritis or IgA nephropathy; cardiovascular diseases or damages preferably selected from cardiac hypertrophy, cardiac remodeling after myocardial infarction, pulmonary congestion and cardiac fibrosis in dilated or in hypertrophic cardiomyopathy, cardiomyopathy such as dilated cardiomyopathy or hypertrophic cardiomyopathy or diabetic cardiomyopathy, left or right ventricular hypertrophy, diabetic myopathy, stroke prevention in congestive heart failure, hypertrophic medial thickening in arteries and/or in large vessels, mesenteric vasculature hypertrophy, or artherosclerosis; renal diseases or damages such as selected from renal hyperfiltration such as after portal renal ablation, proteinuria in chronic renal disease, renal arteriopathy as a consequence of hypertension, nephrosclerosis, hypertensive nephrosclerosis or mesanglial hypertrophy. All these uses have already been described by the applicant in several patent applications relating to the PDGF receptor tyrosine kinase inhibitor “Imatinib”.

A “disease or condition which may be inhibited by PDGF receptor tyrosine kinase inhibitor” is preferably selected from diabetic myopathy, diabetic cardiomyopathy, diabetic nephropathy, autoimmune diseases, atherosclerosis, cardiovascular diseases or damages.

The term “curative” as used herein means efficacy in treating ongoing diseases, disorder or conditions.

The term “prophylactic” means the prevention of the onset or recurrence of diseases, disorders or conditions to be treated.

The term “delay of progression” as used herein means administration of the combination to patients, being in a pre-stage or in an early phase of the disease to be treated, in which patients for example a pre-form of the corresponding disease is diagnosed or which patients are in a condition, e.g. during a medical treatment or a condition resulting from an accident, under which it is likely that a corresponding disease will develop.

The term “combined pharmaceutical preparation” as that term is used herein means that the active ingredients, e.g. imatinib and a DPP-IV inhibitor preferably LAF237, are both administered to a patient as separate entities either simultaneously, concurrently or sequentially with no specific time limits, wherein such administration provides therapeutically effective levels of the two compounds in the body, preferably at the same time. As an example, a non-fixed combination would be two capsules each containing one active ingredient where the purpose is to have the patient achieve treatment with both active ingredients together in the body.

In the present description, the term “treatment” includes both prophylactic or preventative treatment as well as curative or disease suppressive treatment, including treatment of patients at risk of contracting the disease or suspected to have contracted the disease or disorder as well as ill patients. This term further includes the treatment for the delay of progression of the disease.

Preferably, the jointly therapeutically effective amounts of the active agents according to the combination of the present invention can be administered simultaneously or sequentially in any order, e.g. separately (combined pharmaceutical preparation) or in a fixed combination.

Under certain circumstances, drugs with different mechanisms of action may be combined. However, just considering any combination of drugs having different modes of action but acting in the similar field does not necessarily lead to combinations with advantageous effects.

All the more surprising is the experimental finding that the combined administration of a DPP-IV inhibitor and a PDGF receptor tyrosine kinase inhibitor according to the present invention, or, in each case, a pharmaceutically acceptable form thereof, results not only in a beneficial, especially a potentiating or a synergistic, therapeutic effect. Independent thereof, additional benefits resulting from combined treatment can be achieved such as a surprising prolongation of efficacy, a broader variety of therapeutic treatment and surprising beneficial effects on diseases and conditions associated with diabetes (e.g. less gain of weight or less vascular events and cardiovascular morbidity or mortality).

Furthermore when treating diabetes, the combination of the invention is particularly useful for modulating, inhibiting or decreasing or preventing β-cells degeneration, loss of β-cells function, β-cells dysfunction, and/or death of beta cells, such as necrosis or apoptosis of beta cells in a subject with diabetes mellitus (e.g. types I or II) and can also increase of the β-cells mass and the rejuvenation of the β-cells (stimulation of beta-cell growth, differentiation, and cell survival). This effect (especially a potentiating or a synergistic, therapeutic effect of the combination) can be demonstrated by the experimental protocols described in the publication from Pospisilik J A et al. (Diabetes. 2003 March; 52(3):741-50) or in the patent applications US20030220251 and WO0135988.

Accordingly, in a further aspect the present invention concerns the use of a combination comprising
i) a DPP IV inhibitor or a pharmaceutically acceptable salt thereof, and
ii) at least one PDGF receptor tyrosine kinase inhibitor, or a pharmaceutically acceptable salt thereof
   for the manufacture of a medicament for modulating, inhibiting or decreasing or preventing β-cells degeneration, loss of β-cells function, β-cells dysfunction, and/or death of beta cells, such as necrosis or apoptosis of beta cells, for increasing the β-cells mass and for the rejuvenation of the β-cells, especially in a subject with diabetes mellitus (e.g. types I or II).

The invention furthermore relates to a method for modulating, inhibiting or decreasing or preventing β-cells degeneration, loss of β-cells function, β-cells dysfunction, and/or death of beta cells, such as necrosis or apoptosis of beta cells, for increasing the β-cells mass and for the rejuvenation of the β-cells, comprising administering to a warm-blooded animal, including man preferably in a subject with diabetes mellitus (e.g. types I or II), in need thereof a jointly effective amount of a combination of a DPP IV inhibitor or a pharmaceutically acceptable salt thereof with at least one therapeutic agent selected from an agent interacting with a PDGF receptor tyrosine kinase inhibitor, or a pharmaceutically acceptable salt thereof; and at least one additional pharmaceutically acceptable carrier.

The invention furthermore relates to a pharmaceutical composition for modulating, inhibiting or decreasing or preventing β-cells degeneration, loss of β-cells function, β-cells dysfunction, and/or death of beta cells, such as necrosis or apoptosis of beta cells, for increasing the β-cells mass and for the rejuvenation of the β-cells, comprising a combination of a DPP IV inhibitor or a pharmaceutically acceptable salt thereof with at least one PDGF receptor tyrosine kinase inhibitor, or a pharmaceutically acceptable salt thereof; and at least one additional pharmaceutically acceptable carrier.

The diseases, disorders or conditions related to diabetes, particularly type 2 diabetes mellitus, includes but are not limited to diabetic nephropathy, diabetic retinopathy and diabetic neuropathy, macular degeneration, coronary heart disease, myocardial infarction, diabetic cardiomyopathy, myocardial cell death, coronary artery diseases, peripheral arterial disease, stroke, limb ischemia, vascular restenosis, foot ulcerations, endothelial dysfunction and/or atherosclerosis.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

In view of reduced dose of the DPP-IV inhibitor or PDGF receptor tyrosine kinase inhibitor, used according to the present invention, there is a considerable safety profile of the combination making it suitable for first line therapy.

The pharmaceutical composition according to the present invention as described herein before and hereinafter may be used for simultaneous use or sequential use in any order, for separate use or as a fixed combination.

Method or use as described above, wherein the DPP-IV inhibitor and the PDGF receptor tyrosine kinase inhibitor are administered in the form of a combination of the present invention such as a fixed combination or combined preparation or kit of part.

Combination, method or use as described herein, wherein the DPP-IV inhibitor is (S)-1-[(3-hydroxy-1-adamantyl)amino]acetyl-2-cyano-pyrrolidine and wherein the PDGF receptor tyrosine kinase inhibitor is preferably selected from the group consisting of 4-(4-methylpiperazin-1-ylmethyl)-N-[4-methyl-3-(4-pyridin-3-yl)pyrimidin-2-ylamino)phenyl]-benzamide(imatinib), 4-(4-methylpiperazin-1-ylmethyl)-N-[4-methyl-3-(4-pyridin-3-yl)pyrimidin-2-ylamino)phenyl]-benzamide methanesulfonate, 4-Methyl-N-[3-(4-methyl-imidazol-1-yl)-5-trifluoromethyl-phenyl]-3-(4-pyridin-3-yl-pyrimidin-2-ylamino)-benzamide, CT52923 (4-(6,7-dimethoxy-4-quinazolinyl)-N-(3,4-methylenedioxybenzyl)-1-piperazinethiocarboxamide), RP-1776, GFB-111, pyrrolo[3,4-c]-beta-carboline-diones, SU 102 (developed by SUGEN), AG1296 (CAS Number 146535-11-7), AG1296 (CAS Number 71897-07-9) and RPR101511A, or in each case, a pharmaceutically acceptable salt thereof.

Combination, method or use as described above, wherein the DPP-IV inhibitor is (S)-1-{2-[5-cyanopyridin-2yl)amino]ethyl-aminoacetyl)-2-cyano-pyrrolidine and wherein the PDGF receptor tyrosine kinase inhibitor is 4-(4-methylpiperazin-1-ylmethyl)-N-[4-methyl-3-(4-pyridin-3-yl)pyrimidin-2-ylamino)phenyl]-benzamide or, in each case, a pharmaceutically acceptable salt thereof especially 4-(4-methylpiperazin-1-ylmethyl)-N-[4-methyl-3-(4-pyridin-3-yl)pyrimidin-2-ylamino)phenyl]-benzamide methanesulfonate.

According the invention, when the DPP-IV inhibitors, and the PDGF receptor tyrosine kinase inhibitor are administered together, such administration can be sequential in time or simultaneous with, the simultaneous method being generally preferred. For sequential administration, the DPP-IV inhibitor, and the PDGF receptor tyrosine kinase inhibitor can be administered in any order. It is generally preferred that such administration be oral. It is especially preferred that the administration be oral and simultaneous. However, if the subject being treated is unable to swallow, or oral absorption is otherwise impaired or undesirable, parenteral or transdermal administration will be appropriate. When the DPP-IV inhibitor, and the PDGF receptor tyrosine kinase inhibitor are administered sequentially, the administration of each can be by the same method or by different methods.

A further aspect of the present invention is a kit for the prevention of, delay of progression of, treatment of a disease or condition according to the present invention comprising

(a) an amount of a DPP IV inhibitor or a pharmaceutically acceptable salt thereof in a first unit dosage form;

(b) an amount of at least one PDGF receptor tyrosine kinase inhibitor, or, in each case, where appropriate, a pharmaceutically acceptable salt thereof in a second etc. unit dosage form; and

(c) a container for containing said first, second etc. unit forms

In a variation thereof, the present invention likewise relates to a “kit-of-parts”, for example, in the sense that the components to be combined according to the present invention can be dosed independently or by use of different fixed combinations with distinguished amounts of the components, i.e. simultaneously or at different time points. The parts of the kit of parts can then e.g. be administered simultaneously or chronologically staggered, that is at different time points and with equal or different time intervals for any part of the kit of parts. Preferably, the time intervals are chosen such that the effect on the treated disease or condition in the combined use of the parts is larger than the effect that would be obtained by use of only any one of the components.

The present invention thus also relates to a kit of parts comprising

(a) an amount of a DPP IV inhibitor or a pharmaceutically acceptable salt thereof in a first unit dosage form;

(b) an amount of at least one PDGF receptor tyrosine kinase inhibitor or, in each case, where appropriate, a pharmaceutically acceptable salt thereof, in the form of two or three or more separate units of the components (a) to (b), especially for the prevention of, delay of progression of, treatment of a disease or condition according to the present invention.

The invention furthermore relates to a commercial package comprising the combination according to the present invention together with instructions for simultaneous, separate or sequential use.

In a preferred embodiment, the (commercial) product is a commercial package comprising as active ingredients the combination according to the present invention (in the form of two or three or more separate units of the components (a) or (b)), together with instructions for its simultaneous, separate or sequential use, or any combination thereof, in the delay of progression or treatment of the diseases as mentioned herein.

All the preferences mentioned herein apply to the combination, composition, use, method of treatment, “kit of parts” and commercial package of the invention.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubulizing or lyophilizing processes. Thus, pharmaceutical preparations for oral use can be obtained by combining the active compound(s) with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commercially available.

Normally, in the case of oral administration, an approximate daily dose of from about 1 mg to about 360 mg is to be estimated e.g. for a patient of approximately 75 kg in weight.

The dosage of the active compound can depend on a variety of factors, such as mode of administration, homeothermic species, age and/or individual condition.

The pharmaceutical preparation will be supplied in the form of suitable dosage unit form, for example, a capsule or tablet, and comprising an amount, being together with the further component(s) jointly effective, e.g. 50 mg or 100 mg or 150 mg of LAF237.

The pharmaceutical composition according to the present invention as described hereinbefore may be used for simultaneous use or sequential use in any order, for separate use or as a fixed combination.

Thus according to a further embodiment, a DPP-IV inhibitor, is administered with an agent(s) interacting with a PDGF receptor tyrosine kinase inhibitor preferably in the form of a fixed pharmaceutical composition comprising a pharmaceutically acceptable carrier, vehicle or diluent. Accordingly, a DPP-IV inhibitor of this invention, can be administered with a PDGF receptor tyrosine kinase inhibitor as a fixed combination, in any conventional oral, parenteral or transdermal dosage form.

The doses of DPP-IV inhibitor of formula (I) to be administered to warm-blooded animals, for example human beings, of, for example, approximately 70 kg body weight, especially the doses effective in the inhibition of the DPP-IV enzyme, are from approximately 3 mg to approximately 3 g, preferably from approximately 10 mg to approximately 1 g, for example approximately from 20 mg to 200 mg, per person per day, divided preferably into 1 to 4 single doses which may, for example, be of the same size. Usually, children receive about half of the adult dose. The dose necessary for each individual can be monitored, for example by measuring the serum concentration of the active ingredient, and adjusted to an optimum level. Single doses comprise, for example, 10, 40 or 100 mg per adult patient.

The dosage of (S)-1-[(3-hydroxy-1-adamantyl)amino]acetyl-2-cyano-pyrrolidine or 4-Methyl-N-[3-(4-methyl-imidazol-1-yl)-5-trifluoromethyl-phenyl]-3-(4-pyridin-3-yl-pyrimidin-2-ylamino)-benzamide is preferably between 10 and 150 mg daily, most preferably between 25 and 100 mg or 25 and 50 mg daily or between 50 and 150 mg daily. Preferred examples of daily oral dosage are 25, 30, 35, 45, 50, 55, 60, 80, 100 or 150 mg mg. Preferred unit dosage forms comprise 25, 50, 100 or 150 mg of vildagliptin. The application of the active ingredient may occur up to three times a day, preferably one or two times a day.

The preferred herein mentioned PDGF receptor tyrosine kinase inhibitor, will be supplied in the form of suitable dosage unit form, for example, a capsule or tablet, and comprising a therapeutically effective amount, e.g. from about 2 to about 600 mg, as already described herein and in the prior art. The application of the active ingredient may occur up to three times a day, preferably one or two times a day. The same preferred dosage are selected for the fixed combinations.

N-{5-[4-(4-methyl-piperazino-methyl)-benzoylamido]-2-methylphenyl}-4-(3-pyridyl)-2-pyrimidine-amine monomesylate, is preferably administered to a human in a dosage in the range of about 2.5 to 850 mg/day, more preferably 5 to 600 mg/day and most preferably 20 to 300 mg/day. Unless stated otherwise herein, the compound is preferably administered from one to four times per day. Preferred galenic formulations used to deliver imatinib (N-{5-[4-(4-methyl-piperazino-methyl)-benzoylamido]-2-methylphenyl}-4-(3-pyridyl)-2-pyrimidine-amine) or its monomesylate salt are well known in the art e.g. in the patent application WO 03/090720. Preferably imatinib is administered in the form of 50, 10, 200, 300 or 400 mg unit dosage form.

Combination, use or method according to the invention, wherein 50, 100, 200, 300 or 400 mg of a PDGF receptor tyrosine kinase inhibitor selected from the group consisting of 4-(4-methylpiperazin-1-ylmethyl)-N-[4-methyl-3-(4-pyridin-3-yl)pyrimidin-2-ylamino)phenyl]-benzamide, 4-(4-methylpiperazin-1-ylmethyl)-N-[4-methyl-3-(4-pyridin-3-yl) pyrimidin-2-ylamino)phenyl]-benzamide methanesulfonate, 4-Methyl-N-[3-(4-methyl-imidazol-1-yl)-5-trifluoromethyl-phenyl]-3-(4-pyridin-3-yl-pyrimidin-2-ylamino)-benzamide, or, in each case, a pharmaceutically acceptable salt thereof is administered in combination with 50, 100 or 150 mg of (S)-1-[(3-hydroxy-1-adamantyl)amino]acetyl-2-cyano-pyrrolidine or a pharmaceutically acceptable salt thereof is administered to the patient or is administered to the patient daily.

Corresponding doses may be taken, for example, in the morning, at mid-day or in the evening.

Preferably, in case of free combinations, preferred are those dosages for launched products that have been approved and that have been marketed.

Especially preferred are low dose combinations.

To further illustrate the invention, but not by way of limitation, the following examples are provided.

### EXPERIMENTAL PART

The above-cited properties are demonstrable in vitro and in vivo tests, using advantageously mammals, e.g., mice, rats, dogs, monkeys or isolated organs, tissues and preparations thereof. Said compounds can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻⁵ molar and 10⁻¹⁰ molar concentrations. A therapeutically effective amount in vivo may range depending on the route of administration, between about 1 and 500 mg/kg, preferably between about 5 and 100 mg/kg.

In-Vivo Test in Mice for Blood Glucose Control

The above mentioned advantages can be shown by the following experience. ICR-CDI mice (male, five weeks old, body weight: about 20 g) are abstained from food for 18 hours, and then used as test subjects. The combination according to the present invention (e.g. LAF237+imatinib (Glivec®) and the active ingredients alone are suspended in 0.5% CMC-0.14M sodium chloride buffer solution (pH 7.4). The solution thus obtained is administered orally in fixed volume amounts to the test subjects. After predetermined time, the percentage decrease of the blood glucose against the control group is determined.

The Glucose and Insulin Lowering Activity In Vivo can be Evaluated as Follows:

Adult male C57BL ob/ob mice (Jackson Lab, Bar Harbor, Me.) at the age of 11 weeks are housed six per cage in a reversed light cycle room (light on from 6:00 p.m. to 6:00 a.m.) and given access to Purina rodent chow and water ad libitum. On day 1, tail blood samples are taken at 8:00 am and plasma glucose levels are determined. The animals are randomly assigned to the control and compound/combination groups. The means of plasma glucose values of the groups were matched. Animals are then orally dosed with vehicle (0.5% carboxymethyl-cellulose with 0.2% Tween-80) or compounds/combinations (at 30 mg/kg) in vehicle. The mice are dosed daily for a total of 3 days. On day 4, basal blood samples are taken. The plasma samples are analyzed for glucose concentrations using a YSI2700 Dual Channel Biochemistry Analyzer (Yellow Springs Instrument Co., Yellow Springs, Ohio) and insulin concentrations using an ELISA assay.

Beta-Cell Effect

The male Zucker Diabetic Fatty fa/fa (ZDF) rat is a model of Type 2 diabetes. The rats are insulin resistant but normoglycemic from birth and they develop diabetes from about week 7 to week 10 of age. Although the animals are hyperinsulinemic before diabetes onset and during the early stages of diabetes, they later lose glucose-stimulated insulin secretion and finally become almost completely insulinopenic.

The above mentioned advantages can be shown by the following experience which is not limitative. The effects of the therapy with a combination comprising LAF237 and Imatinib (hereinafter: COMBINATION “A”) is studied during a period of time when the animals would normally progress from having impaired glucose tolerance to having overt Type 2 diabetes. Three groups of male ZDF rats (Genetic Models Inc, Indianapolis, Ind., USA) are studied and dosed subcutaneously bi-daily with either vehicle (group A) LAF237 alone, (group B) Imatinib alone, (group C) COMBINATION “A”, n=6 per group. Animals are between 7 and 8 weeks old when dosing is initiated. However, they are elevated compared to a group of non-diabetic Sprague-Dawley rats who had fed glucose levels significantly below the ZDF animals (6.4±0.6 vs 5.8±0.8, mean±SD, p<0.02). This demonstrates the relative impaired glucose tolerant state of the ZDF animals when the study began.

Bromodeoxyuridine (BrDU) is incorporated in newly synthesized DNA and thus will label replicating cells. Six hours before sacrifice the rats are given an injection of 100 mg BrDU/kg intraperitoneally. After sacrifice the pancreata is fixed in 4% PFA, dehydrated, embedded in paraffin, and 3-4 mm sections double stained for BrDU and insulin for the measurement of beta-cell proliferation rate.

Insulin is stained with guinea pig anti-insulin, peroxidase-coupled rabbit anti-guinea pig Ig, and developed with AEC to give a red stain. BrDU is stained by monoclonal mouse anti-BrDU, biotinylated goat anti-mouse Ig, avidin peroxidase, and developed with DAB and CuSO 4 to give a dark brown stain. BrDU stained nuclei of cells with insulin stained cytoplasm is examined in more than 1500 cells per section. The examination of the sections is carried out with the origin of the sections blinded to the observer.

The rats treated with the COMBINATION “A” can show a dose dependent increase in the fraction of beta-cells that had incorporated BrDU as a result of stimulated cell proliferation.

Neighbor sections have to be stained for insulin and the combination of glucagon-somatostatin-pancreatic polypeptide for the measurement of the relative mass of islet beta-cells and nonbeta-cells. The beta-cells are stained for insulin as described above. The nonbeta-cells are stained with a mixture of monoclonal mouse anti-glucagon+rabbit anti-somatostatin+rabbit anti-pancreatic polypeptide, detected by biotinylated swine anti-multible Ig's, avidin peroxidase, and developed with DAB and CuSO 4 to give a dark brown stain. The volume fractions of beta- and nonbeta-cells is estimated by point counting stereologic techniques.

To show that the beta-cell fraction of the total pancreas is significantly higher in the rats given COMBINATION “A” for 6 weeks compared to group A or B treated rats, the-different groups have to be compared together. It can be shown that volume of beta-cells after treatment with the COMBINATION “A” increase at a dose (30 ng/g) where proliferation has not been seen. This experience should support that an inhibition of apoptosis is facilitated by the administered COMBINATION “A”.

Furthermore, specific inhibition of beta-cell apoptosis by the COMBINATION “A”, can be shown in vitro by measuring inhibition of free fatty acid (FFA), glucose, sulfonylurea, or cytokine induced apoptosis in beta cells.

In vitro assays for characterizing the effect of the COMBINATION “A” on the prevention of beta-cell apoptosis induced by FFA: Briefly, pancreatic islet, e.g. rat, mouse and human, isolated and cultured as described in, e.g. Diabetologia 19, 439, 1980; Transplantation, 68, 597, 1999; J. Mol. Med., 77, 93, 1999, Diabetes 48, 1230, 1999, J. Bio. Chem. 274, 18686, 1999; Proc. Natl. Acad. Sci. 95, 2498, 1999;. J. Bio. Chem, 273, 33501, 1998; Diabetologia 42, 55, 1999, with or without 0.1-10 mM long-chain FFAs (2:1 oleate/palmitate; Sigma) and with COMBINATION “A”. Characterization of apoptotic beta cells can be analyzed as described below.

In vitro assays for characterizing the effect of the COMBINATION “A” on the prevention of beta-cell apoptosis induced by glucose or sulfonylureas: Briefly, islets can be isolated and cultured as described in J. Bio. Chem, 273, 33501, 1998, and incubated in 0-30 mM glucose as described in. J. Bio. Chem, 273, 33501, 1998, in order to induce apoptosis. In order to prevent the glucose induced apoptosis the islets can be co-incubated with the COMBINATION “A”.

In vitro assays for characterizing the effect of the COMBINATION “A” on the prevention of beta-cell apoptosis induced by cytokines: Briefly, human and rat islets can be isolated and cultured as described in, e.g. Diabetologia 42, 55, 1999. Cytokine induced apoptosis of rat and human beta cells can be done as describe in Diabetologia 42, 55, 1999. In order to prevent the cytokine induced apoptosis the islets can be co-incubated with the COMBINATION “A”. Characterization of apoptotic beta cells can be analyzed as described below and as described in Diabetologia 42, 55, 1999.

Apoptosis and inhibition thereof can be detected in the following way: The free 3′ OH strand breaks resulting from DNA degradation which is associated with apoptosis can be detected with the terminal deoxynucleotidyl transferase-mediated dUTP-X3′ nick end-labeling (TUNEL) technique (J Cell Biol 199: 493, 1992) or using the following kits e.g. In Situ Cell Death Detection kit; Boehringer Mannheim, Mannheim or ApoTag, Oncor, Gaithersburg, Md.). Preparation of pancreatic sections or islet cultures for apoptosis staining using the TUNEL technique is described in (Diabetologia 42: 566, 1999 and Diabetes 48: 738, 1999).

Alternative experimental protocols are also described in the U.S. Pat. No. 6,890,905 focusing on DPP4 inhibitors, or in WO 2004037277 and WO0101130 A1.

Although the present invention has been described in considerable detail with reference to certain preferred versions thereof, other versions are possible without departing from the spirit and scope of the preferred versions contained herein. All references and Patents (U.S. and others) referred to herein are hereby incorporated by reference in their entirety as if set forth herein in full.

We claim:

1. A pharmaceutical combination composition, comprising:
i) a DPPIV inhibitor or a pharmaceutically acceptable salt thereof, and
ii) a least one PDGF receptor tyrosine kinase inhibitor or a pharmaceutically acceptable salt thereof.

2. The composition according to claim 1, comprising at least one additional pharmaceutically acceptable carrier.

3. The composition according to claim 1, in the form of a combined preparation or a fixed combination.

4. (canceled)

5. A method for the treatment of diseases and disorders that may be inhibited by DPP IV inhibition and/or by inhibiting the PDGF tyrosine kinase receptor, comprising:
administering to a warm-blooded animal in need thereof a jointly effective amount of a combination of a DPP IV inhibitor or a pharmaceutically acceptable salt thereof with at least one PDGF receptor tyrosine kinase inhibitor; and at least one additional pharmaceutically acceptable carrier.

6. The method according to claim 5, wherein the disease or condition is selected from insulin resistance, impaired glucose metabolism (IGT), conditions of impaired glucose tolerance, conditions of impaired fasting plasma glucose, diabetes particularly type 1 or type2 diabetes mellitus, obesity, diabetic retinopathy, macular degeneration, cataracts, diabetic nephropathy, glomerulosclerosis, diabetic neuropathy, erectile dysfunction, premenstrual syndrome, coronary heart disease, hypertension, angina pectoris, myocardial infarction, stroke, vascular restenosis, skin and connective tissue disorders, foot ulcerations and ulcerative colitis, endothelial dysfunction and impaired vascular compliance, and vascular events, cardiovascular morbidity or mortality associated with diabetes or IGT.

7. (canceled)

8. A method for modulating, inhibiting or decreasing or preventing β-cells degeneration, loss of β-cells function, β-cells dysfunction, and/or death of beta cells, such as necrosis or apoptosis of beta cells, for increasing the β-cells mass and for the rejuvenation of the β-cells, comprising administering to a warm-blooded animal, including man preferably in a subject with diabetes, in need thereof a jointly effective amount of a combination of a DPP IV inhibitor or a pharmaceutically acceptable salt thereof with at least one therapeutic agent selected from an agent interacting with a PDGF receptor tyrosine kinase inhibitor, or a pharmaceutically acceptable salt thereof; and at least one additional pharmaceutically acceptable carrier.

9-10. (canceled)

11. The composition according to claim 1, wherein the DPP-IV inhibitor is selected from (S)-1-{2-[5-cyanopyridin-2yl)amino]ethyl-aminoacetyl)-2-cyano-pyrrolidine, vildagliptin, MK-0431 (Sitagliptin), GSK23A, saxagliptin, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide, or in each case, a pharmaceutically acceptable salt thereof.

12. The composition according to claim 11, wherein the DPP-IV inhibitor is (S)-1-{2-[5-cyanopyridin-2yl)amino]ethyl-aminoacetyl)-2-cyano-pyrrolidine or (S)-1-[(3-hydroxy-1-adamantyl)amino]acetyl-2-cyano-pyrrolidine or, in each case, a pharmaceutically acceptable salt thereof.

13. The composition according to claim 1, wherein the PDGF receptor tyrosine kinase inhibitor is selected from the group consisting of 4-(4-methylpiperazin-1-ylmethyl)-N-[4-methyl-3-(4-pyridin-3-yl)pyrimidin-2-ylamino)phenyl]-benzamide (imatinib), 4-(4-methylpiperazin-1-ylmethyl)-N-[4-methyl-3-(4-pyridin-3-yl)pyrimidin-2-ylamino)phenyl]-benzamide methanesulfonate, 4-Methyl-N-[3-(4-methyl imidazol-]-yl)-5-trifluoromethyl-phenyl]-3-(4-pyridin-3-yl-pyrimidin-2-ylamino)-benzamide, CT52923, (4-(6,7-dimethoxy-4-quinazolinyl)-N-(3,4-methylenedioxybenzyl)-1-piperazinethiocarboxamide), RP-1776, GFB-111, pyrrolo[3,4-c]-beta-carboline-diones, SU 102, AG1296, AG1296 and RPR101511A, or in each case, a pharmaceutically acceptable salt thereof.

14. The composition according to claim 13, wherein the PDGF receptor tyrosine kinase inhibitor is preferably selected from the group consisting of 4-(4-methylpiperazin-1-ylmethyl)-N-[4-methyl-3-(4-pyridin-3-yl)pyrimidin-2-ylamino)phenyl]-benzamide (imatintib), 4-(4-methylpiperazin-1-ylmethyl)-N-[4-methyl-3-(4-pyridin-3-yl)pyrimidin-2-ylamino)phenyl]-benzamide methanesulfonate, 4-Methyl-N-[3-(4-methyl-imidazol-1-yl)-5-trifluoromethyl-phenyl]-3-(4-pyridin-3-yl-pyrimidin-2-ylamino)-benzamide, or, in each case, a pharmaceutically acceptable salt thereof.

15. The composition according to claim 1, wherein the DPP-IV inhibitor is (S)-1-[(3-hydroxy-1-adamantyl)amino]acetyl-2-cyano-pyrrolidine or a pharmaceutically acceptable salt thereof, and the PDGF receptor tyrosine kinase inhibitor is selected from the group consisting of 4-(4-methylpiperazin-1-ylmethyl)-N-[4-methyl-3-(4-pyridin-3-yl)pyrimidin-2-ylamino)phenyl]-benzamide (imatinib), 4-(4-methylpiperazin-1-ylmethyl)-N-[4-methyl-3-(4-pyridin-3-yl)pyrimidin-2-ylamino)phenyl]-benzamide methanesulfonate, 4-Methyl-N-[3-(4-methyl-imidazol-1-yl)-5-trifluoromethyl-phenyl]-3-(4-pyridin-3-yl-pyrimidin-2-ylamino)-benzamide, or, in each case, a pharmaceutically acceptable salt thereof.

16. The method according to claim 5, wherein
50 to 600 mg of a PDGF receptor tyrosine kinase inhibitor selected from the group consisting of 4-(4-methylpiperazin-1-ylmethyl)-N-[4-methyl-3-(4-pyridin-3-yl) pyrimidin-2-ylamino)phenyl]-benzamide, 4-(4-methylpiperazin-1-ylmethyl)-N-[4-methyl-3-(4-pyridin-3-yl)pyrimidin-2-ylamino)phenyl]-benzamide methanesulfonate, 4-Methyl-N-[3-(4-methyl-imidazol-1-yl)-5-trifluoromethyl-phenyl]-3-(4-pyridin-3-yl-pyrimidin-2-ylamino)-benzamide, or, in each case, a pharmaceutically acceptable salt thereof and
between 25 and 150 mg of (S)-1-[(3-hydroxy-1-adamantyl)amino]acetyl-2-cyano-pyrrolidine or a pharmaceutically acceptable salt thereof is administered to the patient daily.

17. The composition according to claim 1, wherein
50 to 600 mg of a PDGF receptor tyrosine kinase inhibitor is in the composition and the PDGF receptor tyrosine kinase inhibitor is selected from the group consisting of 4-(4-methylpiperazin-1-ylmethyl)-N-[4-methyl-3-(4-pyridin-3-yl)pyrimidin-2-ylamino)phenyl]-benzamide, 4-(4-methylpiperazin-1-ylmethyl)-N-[4-methyl-3-(4-pyridin-3-yl)pyrimidin-2-ylamino)phenyl]-benzamide methanesulfonate, 4-Methyl-N-[3-(4-methyl-imidazol-1-yl)-5-trifluoromethyl-phenyl]-3-(4-pyridin-3-yl-pyrimidin-2-ylamino)-benzamide, or, in each case, a pharmaceutically acceptable salt thereof and
between 25 and 150 mg of (S)-1-[(3-hydroxy-1-adamantyl)amino]acetyl-2-cyano-pyrrolidine or a pharmaceutically acceptable salt thereof is used in the composition.

18. The composition according to claim 1, wherein
the PDGF receptor tyrosine kinase inhibitor is selected from the group consisting of 4-(4-methylpiperazin-1-ylmethyl)-N-[4-methyl-3-(4-pyridin-3-yl)pyrimidin-2-ylamino)phenyl]-benzamide, 4-(4-methylpiperazin-1-ylmethyl)-N-[4-methyl-3-(4-pyridin-3-yl) pyrimidin-2-ylamino)phenyl]-benzamide methanesulfonate, 4-Methyl-N-[3-(4-methyl-imidazol-1-yl)-5-trifluoromethyl-phenyl]-3-(4-pyridin-3-yl-pyrimidin-2-ylamino)-benzamide, or, in each case, a pharmaceutically acceptable salt thereof and
the DPPIV inhibitor is (S)-1-[(3-hydroxy-1-adamantyl)amino]acetyl-2-cyano-pyrrolidine or a pharmaceutically acceptable salt thereof,
wherein an amount of the PDGF receptor tyrosine kinase inhibitor is selected from the group consisting of 50, 100, 200, 300 and 400 mg per day and an amount of DPPIV inhibitor is selected from the group consisting of 50, 100 and 150 mg per day.

19. The composition according to claim 1, wherein
the PDGF receptor tyrosine kinase inhibitor is selected from the group consisting of 4-(4-methylpiperazin-1-ylmethyl)-N-[4-methyl-3-(4-pyridin-3-yl)pyrimidin-2-ylamino)phenyl]-benzamide, 4-(4-methylpiperazin-1-ylmethyl)-N-[4-methyl-3-(4-pyridin-3-yl)pyrimidin-2-ylamino)phenyl]-benzamide methanesulfonate, 4-Methyl-N-[3-(4-methyl-imidazol-1-yl)-5-trifluoromethyl-phenyl]-3-(4-pyridin-3-yl-pyrimidin-2-ylamino)-benzamide, or, in each case, a pharmaceutically acceptable salt thereof and
the DPPIV inhibitor is (S)-1-[(3-hydroxy-1-adamantyl)amino]acetyl-2-cyano-pyrrolidine or a pharmaceutically acceptable salt thereof,
wherein an amount of the PDGF receptor tyrosine kinase inhibitor is selected from the group consisting of 50, 100, 200, 300 and 400 mg per day and an amount of DPPIV inhibitor is selected from the group consisting of 50, 100 and 150 mg per day.

20. (canceled)
